(19) United States
(12) Patent Application Publication
(10) Pub. No.: US 2015/0018881 A1
(43) Pub. Date: Jan. 15, 2015

(54) KNOTLESS INTERFACE FOR THREADED ANCHOR

(71) Applicant: DePuy Mitek, LLC, Raynham, MA (US)

(72) Inventors: Nathan Cauldwell, Attleboro, MA (US); Douglas W. Dunn, Mansfield, MA (US)

(21) Appl. No.: 14/503,448

(22) Filed: Oct. 1, 2014

Related U.S. Application Data
(62) Division of application No. 12/120,293, filed on May 14, 2008, now abandoned.

(51) Int. Cl. A61B 17/04; A61F 2/08

(52) U.S. Cl. CPC A61B 17/0401; A61F 2/0811; A61B 2017/0409; A61B 2017/0414; A61B 2017/044; A61B 2017/0464; A61F 2002/0817
USPC 606/232

(57) ABSTRACT

Devices and methods for anchoring tissue to bone are provided. In one embodiment, a suture anchor is provided that includes a bone screw and a collar that can be configured to rotate independent of the bone screw. In one aspect, the collar can be rotatably disposed on a cylindrical seating portion of the bone screw. The collar can include an eyelet and a hook that is angularly offset from the eyelet. In use, a suture loop can be fixedly attached to the collar through the eyelet.

### FIELD OF THE INVENTION

The present invention relates to surgical fasteners, e.g, knotless suture anchor systems that secure soft tissue to bone, and methods of using the same.

### BACKGROUND OF THE INVENTION

Soft tissues, such as ligaments, tendons and muscles, are attached to a large portion of the human skeleton. In particular, many ligaments and tendons are attached to the bones which form joints, such as shoulder and knee joints. A variety of injuries and conditions require attachment or reattachment of a soft tissue to bone. For example, when otherwise healthy tissue has been torn away from a bone, surgery is often required to reattach the tissue to the bone to allow healing and a natural reattachment to occur.

A number of devices and methods have been developed to attach soft tissue to bone. These include screws, staples, cement, suture anchors, and sutures alone. Some of the more successful methods involve use of a suture anchor to attach a suture to the bone, and tying the suture in a manner that holds the tissue in close proximity to the bone.

The tissue may be attached to the bone during traditional open surgery, or during minimally invasive (e.g., arthroscopic) surgical procedures. Minimally invasive surgical procedures are preferred since they are less invasive and are less likely to cause patient trauma. In a minimally invasive surgical procedure, the surgeon performs diagnostic and therapeutic procedures at the surgical site through small incisions, called portals, using instruments specially designed for this purpose. One problem encountered in these less invasive surgical procedures is that the surgeon has significantly less room to perform the required manipulations at the surgical site and the surgeon's hands are remote from the surgical site. Thus, devices and methods are needed which will allow a surgeon to effectively and easily attach tissue to bone in the small spaces provided by less invasive surgical procedures.

Conventional methods for attaching soft tissue to bone typically require that the surgeon tie a knot in the suture thread to attach the suture to an anchor, or to attach the tissue to the bone using the suture. This knotting process can be difficult and tedious, particularly during arthroscopic procedures where the surgeon must remotely manipulate the suture using tools inserted through an endoscopic tube. Complicating the procedure is the fact that multiple knots must often be tied. In some cases, knots and other bulky attachment means can irritate tissue over time. These knots may also “stand proud” above the tissue and interfere with movement and healing.

Although some knotless suture anchor designs are known, there remains a need for reliable and easy-to-use suture anchors that do not require surgeons to form one or more knots with a suture.

### SUMMARY OF THE INVENTION

Devices and methods for anchoring tissue to bone are provided herein. In general, the devices and methods described below provide a surgeon with the ability to attach soft tissue to bone using a suture without the need to tie a knot in the suture to attach the suture to the anchor or to the tissue. The devices and methods also allow a surgeon to effectively and easily attach tissue to bone in the small spaces provided by less invasive surgical procedures.

Various aspects of such a suture anchor are provided herein. In a first aspect, the suture anchor includes a bone screw and a collar that is associated with the bone screw. As will be described, the bone screw has a major diameter, a minor diameter, and a helical thread, and the collar can have a hook and an eyelet that is effective to receive a length of suture. In one aspect, the eyelet can be angularly offset from the hook at any angle, such as about a 180 degree angle or less. In another exemplary embodiment, the bone screw may include a cylindrical seating portion for receiving the collar that can have a diameter less than the minor diameter of the screw. For example, the cylindrical seating portion can be disposed at a proximal portion of the bone screw distal of a head of the bone screw. In another exemplary embodiment, the cylindrical seating portion is disposed between adjacent thread crests. In yet another exemplary embodiment, the collar can be disposed at the root of the helical thread. As will also be described, the collar can be configured to rotate independent of the bone screw. For example, it can be rotatably disposed on the bone screw such as on the cylindrical seating portion. The suture anchor can be constructed such that a maximum dimension of a footprint of the collar is less than or equal to the major diameter of the bone screw. The hook can be any element capable of capturing a suture loop. For example, the hook can include a U-shaped member having a distal-faced opening.

In another aspect the suture anchor includes a bone screw and a collar rotatably disposed on the bone screw. Further, the suture anchor can include a hook formed on a portion of the collar. In an exemplary embodiment, the suture anchor also includes a suture loop fixedly attached to a portion of the collar. For example, the suture loop can be fixedly attached to the collar through an eyelet disposed on the collar, the eyelet being angularly offset from the hook. Also, similar to those embodiments summarized above, the bone screw has a major diameter, a minor diameter, and a helical thread that has a crest and a root. The bone screw can also include cylindrical seating portion that has a diameter less than the minor diameter of the screw. In one embodiment, the collar can be rotatably seated within the cylindrical seating portion. In another embodiment, the collar can be disposed at the root of the helical thread.

Various aspects of a method for anchoring tissue to bone are also provided herein. In one such aspect, the method includes providing a suture anchor having a collar rotatably disposed thereon with a suture loop pre-attached to a portion of the collar and a utility suture loop attached to the suture loop. The method also includes passing the utility suture through a detached segment of tissue and manipulating the suture anchor to engage a portion of the suture loop within a portion of the collar. The suture anchor can then be rotated to implant it into bone while tensioning the suture loop to prevent the collar from rotating with respect to the suture anchor such that the suture loop reattaches the detached segment to bone. The method can also include attaching a needle to the utility suture. Similar to the aspects disclosed above, the suture loop can be pre-attached to an eyelet on the collar. The suture loop can also be engaged with a hook portion of the collar.

### BRIEF DESCRIPTION OF THE DRAWINGS

The invention will be more fully understood from the following detailed description taken in conjunction with the accompanying drawings, in which:

FIG. 1 is a perspective view of an embodiment of a suture anchor according to the present invention;

FIG. 2 is an elevation view of the suture anchor of FIG. 1;

FIG. 3 is a perspective view of the bone screw with the collar removed;

FIG. 4 is an elevation view of the bone screw with the collar removed;

FIG. 5 is a perspective sectional view of the suture anchor taken proximal to the collar;

FIG. 6 is a perspective view of the collar;

FIG. 7 is a top view of the collar of FIG. 6;

FIG. 8 is a perspective view of the suture anchor of FIG. 1 showing the suture loop and the utility suture.

FIG. 9 is a perspective view of the suture anchor of FIG. 1 showing the suture loop engaged with the hook.

FIGS. 10a-10c illustrate the procedure for the attachment of tissue to bone according to the present invention.

FIGS. 11a-11c illustrate another procedure for the attachment of tissue to bone according to the present invention.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for anchoring tissue to bone. As summarized above, the presently disclosed embodiments provide a surgeon with the ability to attach soft tissue to bone using a suture without the need to tie a knot in the suture to attach the suture to the anchor or to the tissue. The devices and methods also allow a surgeon to effectively and easily attach tissue to bone in the small spaces provided by less invasive surgical procedures. More specifically, the suture anchor provided herein includes a bone screw and a collar rotatably disposed on a portion of the bone screw. A suture loop can be attached to the collar and a length of utility suture can be attached to the suture loop. In use, one advantage provided by this design is the reduced tendency of the suture attached to the bone anchor to twist or tangle during a procedure. For example, a surgeon can begin by pulling the suture loop through a piece of tissue using a utility suture. The surgeon can then engage the suture loop to a portion of the collar, such as a hook, thereby attaching the tissue to the suture anchor at two points. The suture anchor can then be inserted into bone or a bone hole and rotated to advance it into bone or a bone hole. During insertion of the anchor, the surgeon can apply tension to the utility suture to prevent the collar from rotating with respect to the suture anchor. As the bone screw is driven into the bone, the collar and attached suture can be driven below the bone surface, thereby anchoring the attached tissue to the bone. The depth of insertion of the suture anchor can be used to control the degree of tension applied to the tissue.

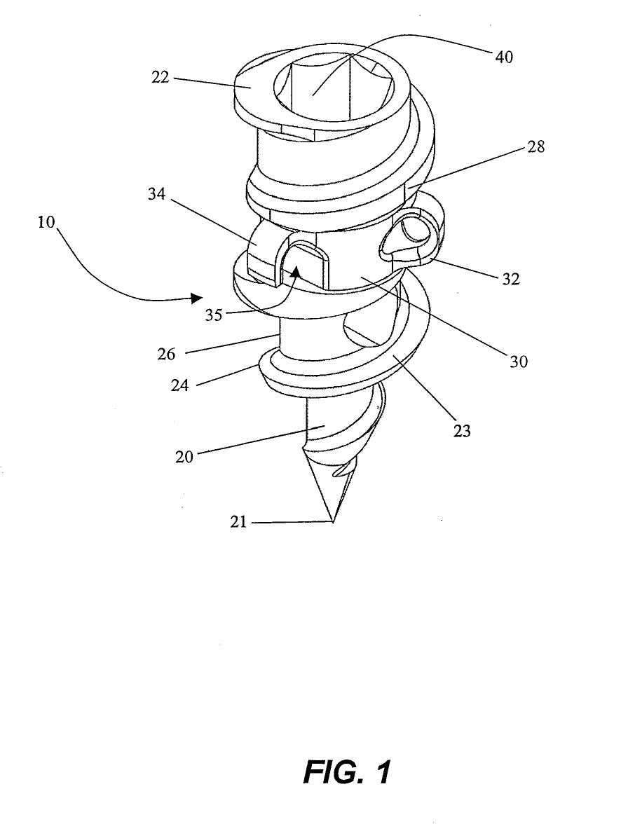

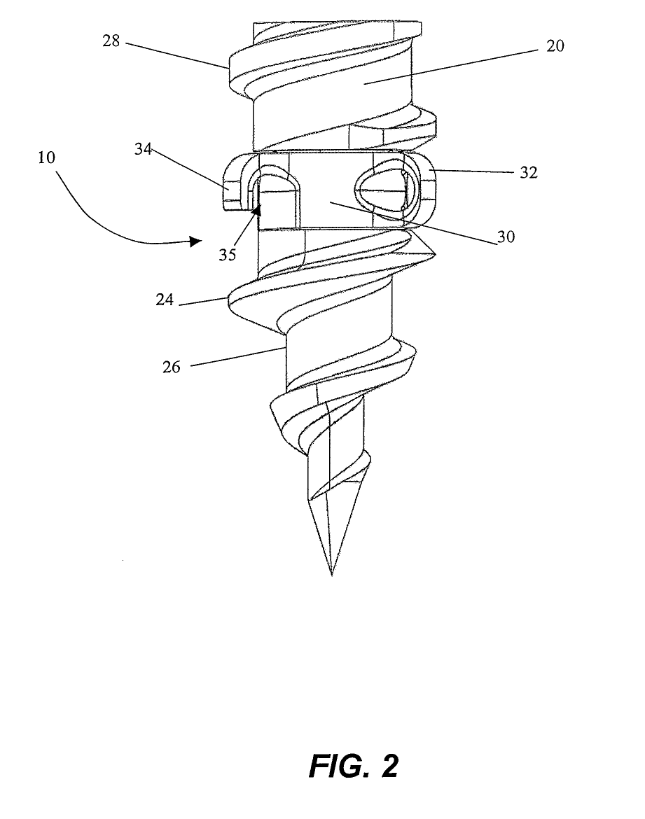

Referring now to FIGS. 1 and 2, a suture anchor according to the present invention is shown. The suture anchor 10 includes a bone screw 20 and a collar 30. As shown, the bone screw 20 has a distal end 21 and a proximal end 22. The bone screw 20 also includes a helical thread 23, which can be formed in any configuration and has a crest 24 and a root 26. An Allen or other female socket 40 can be formed in the proximal end 22 to receive a driver (not shown) so that torque can be applied along the axis of the screw 20 to insert the screw 20 into bone. The collar 30, which can be rotatably disposed on the bone screw 20, includes an eyelet 32 and a hook 34.

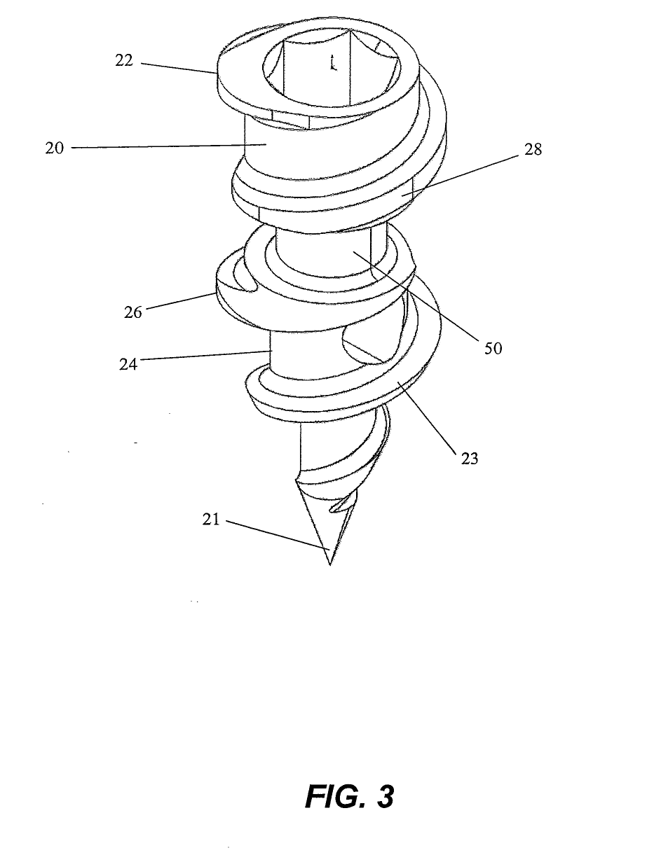

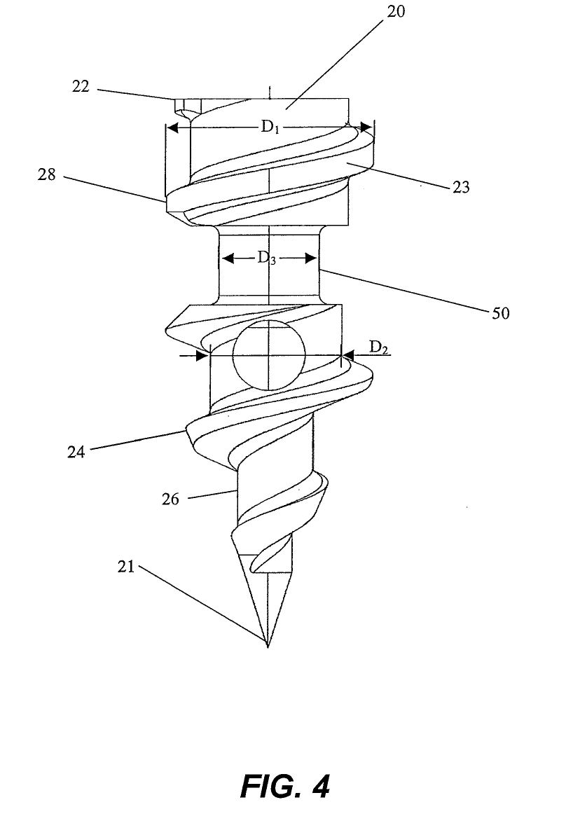

FIGS. 3 and 4 provide an exemplary embodiment of the presently disclosed bone screw 20. One skilled in the art will readily appreciate that the dimensions of the bone screw 20 may vary depending upon the desired surgical applications and/or the patient's anatomy.

As shown in the exemplary embodiment of FIGS. 3 and 4, the bone screw 20 includes a helical thread 23 with a configuration such that a major diameter (D₁) and a minor diameter (D₂) of the screw 20 can increase from the distal end 21 to the proximal end 22 of the bone screw 20. In another embodiment (not shown), the major and minor diameters of the screw 20 can remain substantially uniform from the distal end 21 to the proximal end 22 of the bone screw. In the exemplary embodiment of FIGS. 3 and 4, the thread 23 begins proximal to the distal end 21 and proceeds along substantially the entire length of the bone screw 20, interrupted by the cylindrical seating portion 50. One skilled in the art will appreciate that the thread 23 can be configured to run clockwise or counter-clockwise.

The helical thread can include various sizes and/or dimensions. In an exemplary embodiment, the threads have a buttress cross-section, and a substantially constant thread crest height, thickness, and pitch along the length of the screw. Further, the thread depth relates to bony purchase and correlates to screw extraction strength. Such features can be optimized for stabilization of the bone screw within the bone. As shown, a portion 28 of the crest 24 of the helical thread 23 proximal to the collar can be blunt. For example, the blunt portion can be formed as a flattened surface at the crest 24 of the thread 23. As will be discussed below, the blunt portion 28 can prevent the thread from weakening or damaging a suture.

The bone screw 20 can optionally include a cylindrical seating portion 50 that has a diameter (D₃) less than the minor diameter of the screw. The cylindrical seating portion 50 can be disposed at any location between the distal end 21 and proximal end 22 of the bone screw 20. For example, the cylindrical seating portion 50 can be disposed at a proximal portion of the bone screw, distal of a head of the bone screw. As will be discussed in more detail below, the smaller diameter of the cylindrical seating portion 50 provides one way in which the collar 30 (FIGS. 1 and 2) can be rotatably disposed on the cylindrical portion 50. One benefit of the rotatability of the collar 30 with respect to the screw 20 is that the bone screw can be threaded into bone by rotation while the collar 30 remains in a fixed position, such as by tension applied to suture that is attached to the collar.

In the exemplary embodiment shown in FIGS. 3 and 4, the cylindrical seating portion 50 is disposed between the distal end 21 and the proximal end 22 of the bone screw 20. The cylindrical seating portion 50 can be formed in various shapes and diameters. For example, the cylindrical seating portion can have a diameter (D₃) less than the minor diameter (D₂) of the screw 20. In such an exemplary embodiment, the diameter of the cylindrical seating portion 50 can be optimized to allow the collar 30 (FIGS. 1 and 2) to rotate with respect to the bone screw when the collar 30 is disposed thereon. However, the relationship between the inner diameter of the collar 30 and the cylindrical seating portion 50 should be such that sufficient space is provided to permit rotation of the collar while minimizing undesirable motion of the collar 30 perpendicular to the longitudinal axis of the screw 20. The length of the cylindrical seating portion 50 along the axis of the bone screw 20 can also vary. For example, the length of the cylindrical seating portion 50 can be approximately the same as or slightly greater than the length of the collar so as to minimize undesirable motion of the collar 30 parallel to the axis of the screw 20 (i.e., sliding, snagging, or toggling), while allowing the collar to be free to rotate to rotate with respect to the bone screw.

In another embodiment, the collar 30 can be rotatably seated on a portion of the bone screw 20 other than a cylindrical seating portion. For example, the collar 30 can be disposed at the root 26 of the helical thread 23. In such an embodiment (not shown), the helical thread 23 may optionally be modified to provide a location for the collar 30 to be positioned on the screw so that it can rotate around an axis parallel to the longitudinal axis of the bone screw 20.

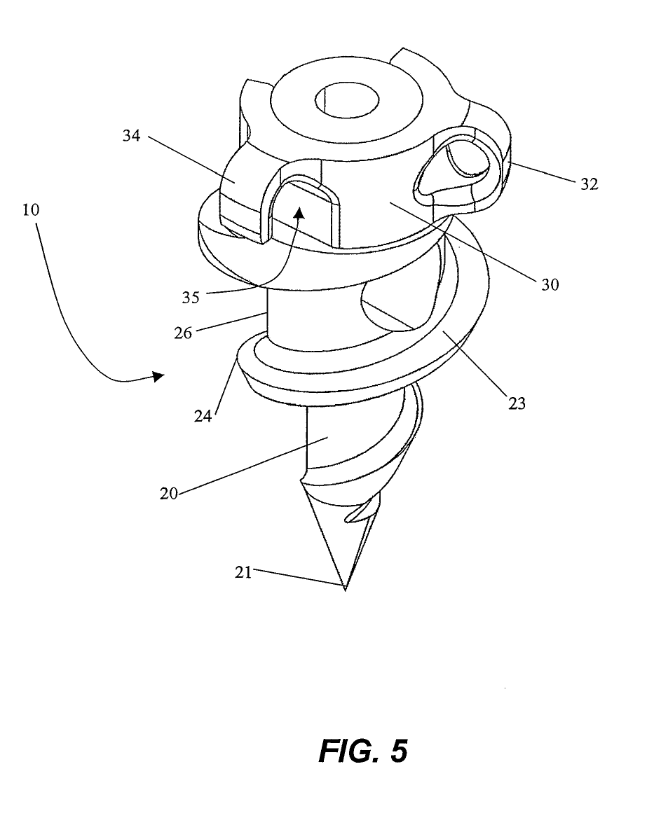

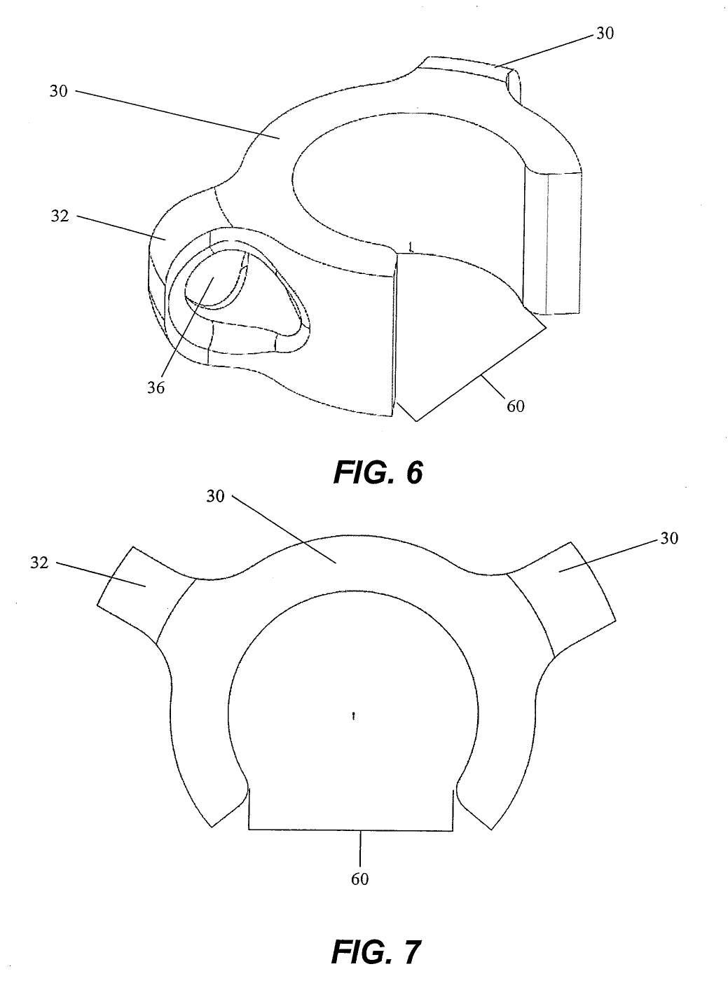

FIG. 5 shows a view of the anchor 10 truncated (for descriptive purposes) just proximal to the collar 30. As shown in FIG. 5, a portion of the collar 30 is formed in a generally circular shape, and it can be attached to the bone screw 30 using any means. For example, the collar 30 can be formed with an opening 60, as shown in FIGS. 6 and 7. In such an exemplary embodiment, the collar 30 may be substantially C-shaped. The opening 60 allows the collar 30 to deform slightly so that it can be snap fit onto the bone screw 20, for example onto the cylindrical seating portion 50.

The collar 30 can include a capture mechanism, such as, for example, a hook 34 (FIGS. 1, 2, and 5). However, the capture mechanism can be any feature capable of capturing a loop of suture, such as a peg, a knob, or a clip. Those skilled in the art will appreciate that a variety of other such shapes can be employed to capture a suture loop. As shown in FIGS. 1, 2, and 5, the hook 34 is formed integrally with the collar 30 and has a distal facing opening 35. As discussed below, the distal facing opening 35 allows a suture loop to be captured or received by the hook 34.

The collar 30 can also include a suture attachment point, such as, for example, an eyelet 32, which allows a suture loop to be fixedly attached to the collar 30. As shown in FIGS. 5-7, the eyelet 32 can be formed integrally with the collar 30, such that it includes an opening 36. The opening 36 is sized to allow a suture loop of a predetermined diameter to be passed through the opening 36, while preventing the passage through the opening 36 of a knot or other feature formed in or disposed on the suture loop.

In one embodiment, the collar 30, including attachment points such as eyelet 32 and hook 34, has a footprint that has a maximum dimension that is less than the outer diameter of the helical thread 23, as shown for example in FIGS. 1, 2, 8, and 9. In such a configuration, the collar 30 can be driven below the surface of a patient's bone, anchoring the suture to the bone. In an alternative embodiment (not shown), the footprint of the collar 30, including attachment points such as eyelet 32 and hook 34, can be slightly greater than the outer diameter of the helical thread 23. However, one skilled in the art will readily appreciate that the dimensions of the collar 30 may also vary depending upon the desired surgical applications. For example, the inner and outer diameter of the collar 30 and the dimensions of the eyelet 32 and hook 34 can vary and will typically depend on the nature of the procedure and/or the patient's anatomy.

The eyelet 32 and hook 34 can be angularly spaced from each other on the collar 30 by any angular displacement, which can vary depending upon desired surgical applications. For example, the eyelet 23 and hook 34 can be spaced from each other by about 180 degrees, i.e., on opposite sides of the collar 30. As shown in FIG. 7, the eyelet 32 alternatively can be spaced from the hook 34 by less than 180 degrees. For example, an angular spacing of less than about 180 degrees such as, for example, about 120 degrees, allows for a suture loop to be attached to the same side of the bone screw 20 during insertion of the bone screw 20 into bone.

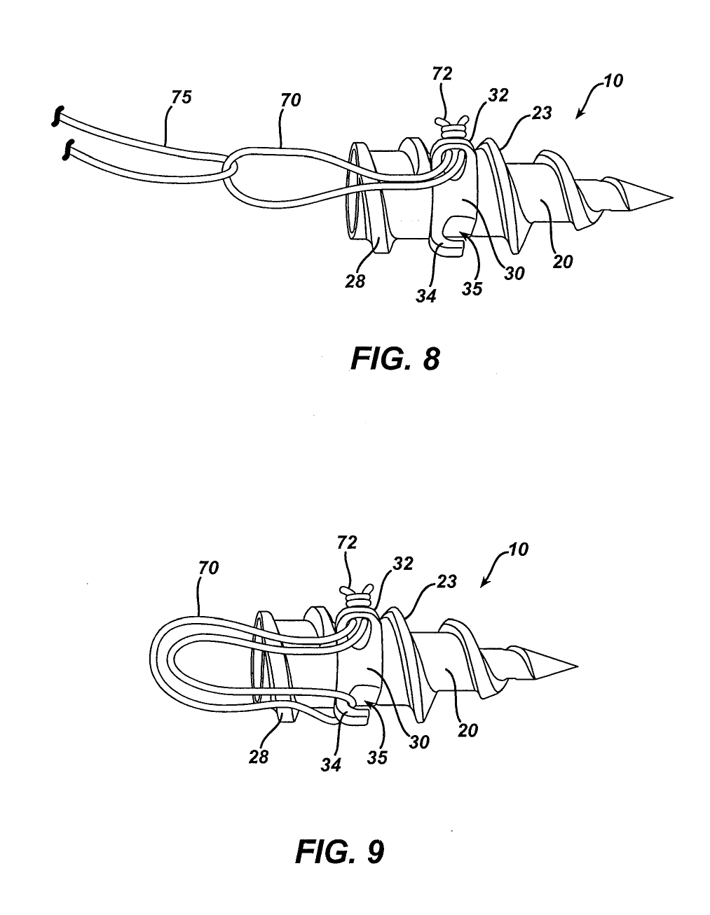

As will be discussed in more detail below, the presently disclosed embodiments provide a surgeon with the ability to attach soft tissue to bone using a suture without the need to tie a knot in the suture to attach the suture to the anchor or to the tissue. Referring now to FIGS. 8 and 9, a suture loop 70 may formed by a mechanical binding device, such as a clip or clasp, or by tying the two free ends of a suture thread to form a knot 72. The suture loop 70 can also be formed using any conventional bonding method such as heat welding, ultrasonic welding, etc. The suture loop can be provided separately from the suture anchor or it may be pre-attached to the suture anchor. In either case, a set of suture loops of varying lengths can be provided with the suture anchor to allow a surgeon to select the desired length of suture loop that is suitable for a given procedure or patient. A surgeon can also form a suture loop of a desired length using suture not provided with the suture anchor.

The suture loop 70 can be attached to the suture anchor 10 by passing the suture loop through an eyelet 32 formed on the collar 30. The dimensions of the eyelet 32 can be such that the knot 72 or other mechanical object or device on the suture loop 70 cannot pass through the eyelet 32, thereby securing the suture loop 70 to the collar 30. If the method used to form the suture loop results in a smaller knot or other joint than the opening in the eyelet, then an additional device or object, such as a bead or clip, can be used to prevent the suture loop from passing through the eyelet. In an alternative embodiment (not shown), the suture loop can be formed by passing one end of a length of suture through the eyelet 32 and then joining it to the other end of the length of suture, forming a suture loop that is joined to the eyelet. In this embodiment, the ends of the suture loop may be joined by any means, such as a knot, a clip or clasp, or any conventional bonding method such as heat welding, ultrasonic welding, etc.

FIG. 8 also shows a length of suture 75, used as a utility suture, that can be threaded through the suture loop 70. As will be discussed in more detail below, the utility suture loop 75 can be used to manipulate the suture loop 70. For example, the utility suture 75 can be used to pull the suture loop 70 through a detached segment of tissue. The utility suture 75 can also be used to tension the suture loop 70 during insertion of the suture anchor 10 into bone to prevent the collar 30 from rotating.

The suture loop 70 and utility suture 75 may be constructed from thread suitable for use as a suture. A variety of suture materials are well known to those of skilled in the art. Exemplary materials include braided polyester and polydioxanone (PDS). The length of the suture loop 70 and utility suture 75 may be determined by a person of skilled in the art, depending upon the desired surgical application. This dimension depends, to a large extent, upon the dimensions of the tissue to be attached, the type of surgery to be performed, and whether an open or minimally invasive (e.g., arthroscopic) surgical technique is to be used. By way of example, the length of the suture loop 70 may range from about 5 mm to about 20 mm.

The various embodiments of the suture anchor described herein can be used in methods for reattaching and anchoring soft tissue to bone. The method of the present invention is useful in various surgical procedures, and is applicable to both open and minimally invasive (e.g., arthroscopic) procedures. Examples of the specific procedures to which the present invention is applicable include, but are not limited to the following open and arthroscopic shoulder surgeries: rotator cuff repair, Bankart repair, SLAP lesion repair, capsule shift repair (glenoid rim). Open surgical procedures for the shoulder to which the invention is also applicable include capsule shift/capsulo-labral reconstruction at the anterior glenoid rim site, capsule shift/capsulo-labral reconstruction at the lesser tuberosity of the humerus, biceps tenodesis, and acomio-clavicular separation. Other surgical procedures to which the invention is applicable include biceps tendon reattachment, Achilles tendon repair/reattachment, lateral stabilization of the ankle, medial stabilization at the medial talus site of the ankle, Hallux Valgus reconstruction of the foot, medial collateral ligament repair, lateral collateral ligament repair, joint capsule closure to anterior proximal tibia, posterior oblique ligament or joint capsule to tibia repair, extra capsular reconstruction/ITB tenodesis, and patellar ligament and tendon avulsion repair.

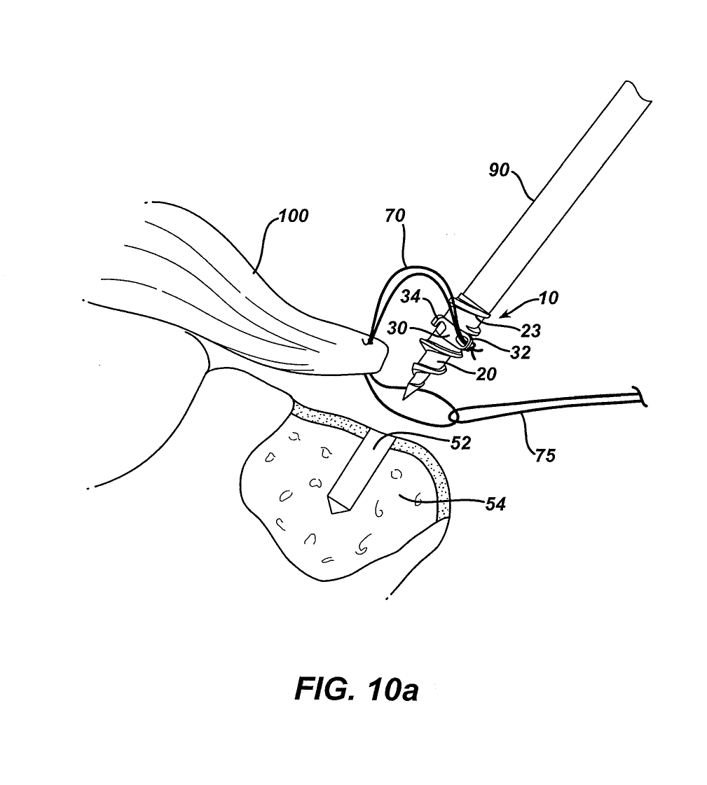

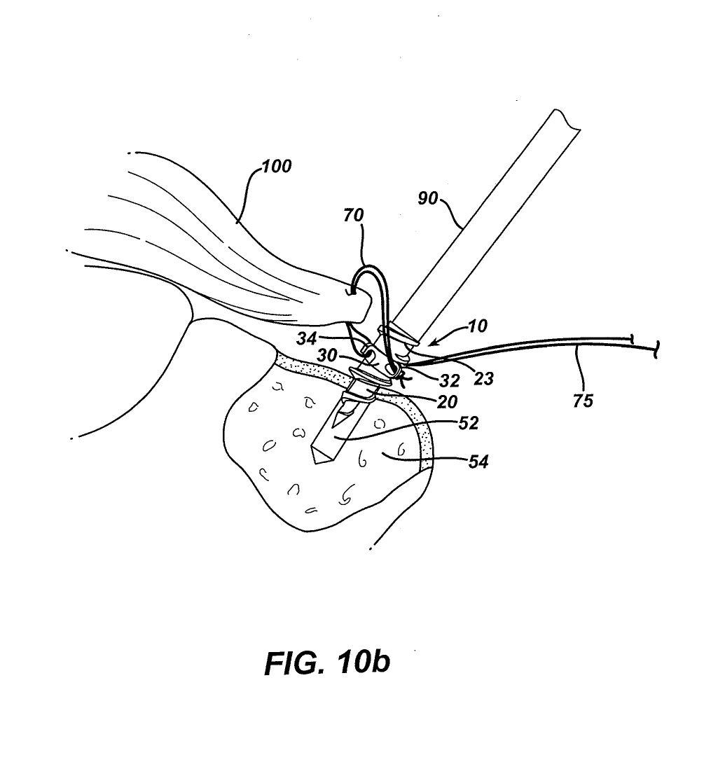

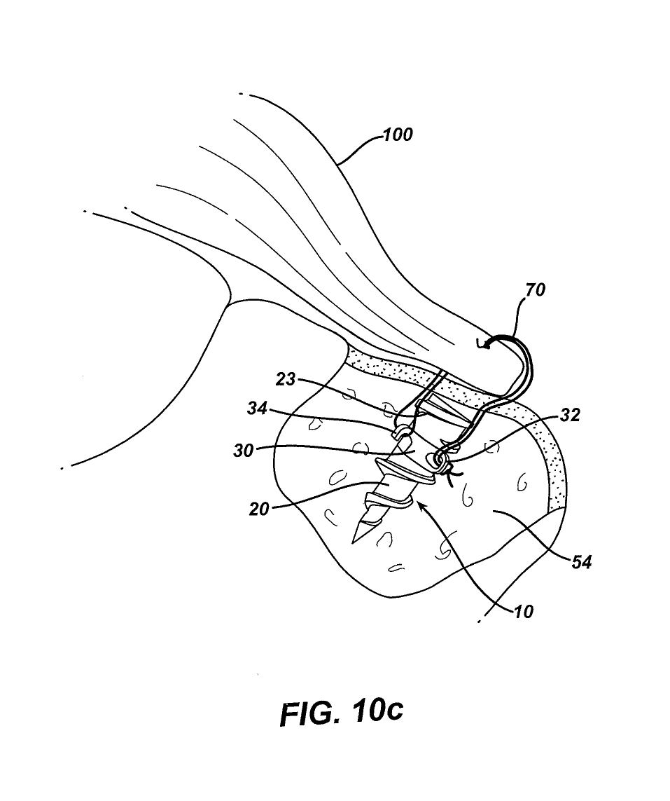

In an exemplary embodiment, the method includes providing a suture anchor of the type described above and illustrated in FIGS. 1-9 having a first suture loop 70 attached to eyelet 32 and a utility suture 75 attached to suture loop 70. The surgical procedure can begin by forming a minimally invasive percutaneous incision through the tissue located adjacent to the desired surgical site. One skilled in the art will readily appreciate that the location, shape, and size of the incision will depend on the nature of the procedure, the patient's anatomy, and/or the preference of the surgeon. Following the formation of an incision to provide access to the surgical site a bore 52 can be formed in a bone 54, as shown in FIG. 10a. One skilled in the art will appreciate that the anchor 10 can be inserted into bone without the need for a bore to be formed in the bone. For example, the bore 52 can be optional if the threads of the bone screw are self-threading. If a bore 52 is used, the diameter of the bore 52 should be slightly smaller than the outer diameter of the helical thread 23 at the proximal end of the bone screw 20. In an exemplary embodiment, the diameter of the bore 52 is in the range of approximately 2 mm to 5 mm when the outer diameter of the helical thread 23 at the proximal end of the bone screw 20 is about 3 mm to about 7 mm. If a bore 52 is used, the length of the bore 52 should be of sufficient length to allow the anchor to be driven into the bone 54, and to enable the depth of the anchor to be adjusted to help control the tightness of the suture loop 70. The actual length of the bore 52 will depend upon the length of the suture loop 70, the thickness of the detached tissue 100, and the configuration of the bone screw 20.

To reach the configuration shown in FIG. 10a, the utility suture 75 and the attached suture loop 70 are passed through the detached tissue 100 to advance the interlocked suture loop 70 through the tissue. If the procedure is being performed arthroscopically, the utility suture 75 and the tool with which it is associated will be pulled from, and exit through, an exit portal (not shown). Those skilled in the art will appreciate that other methods of passing the utility suture 75 and suture loop 70 through the detached tissue 100 can be used depending upon the desired surgical application.

In FIG. 10a, the suture loop 70 is positioned near the bore 52 by manipulating the position of the utility suture 75. When the suture loop 70 is in its desired position, the insertion tool 90 and the attached suture anchor 10 are maneuvered so that a portion of the suture loop 70 is engaged, received or captured by a portion of the collar, for example, by the distal facing opening of hook 34 on the collar 30 of suture anchor 10, as shown in FIG. 10b. After the suture loop 70 is engaged by the hook 34, the anchor 10 is aligned with the optional bore 52. The suture anchor 10 can then be driven into the bore 52, for example by rotating the insertion tool 90. During the insertion of the suture anchor 10, the utility suture 75 can be used to provide tension on the suture loop 70, which prevents the collar 30 from rotating even though the bone screw 20 is being rotated for insertion. Limiting, eliminating, or controlling rotation of the collar 30 with respect to the bone screw 20 can be desirable as it avoids tangling of the suture loop 70 and/or wrapping the suture loop 70 around the shaft of the bone screw 20 during insertion. As the anchor 10 is driven into the bone 54, the collar 30 and attached suture loop 70 can be driven below the surface of the bone 54. During this operation, the suture loop 70 will become trapped between the threads of the bone screw and the bone 54. For example, if a bore 52 is used, the suture loop will become trapped between the threads of the bone screw 20 and the walls of the bore 52. As discussed above, a portion of the crest of the helical thread 23 proximal to the collar can be blunt to avoid damage to the suture 70 as the bone screw 20 rotates into the bone 54.

Referring to FIGS. 10b and 10c, when the suture anchor 10 is advanced into the bone 54, the thread 23 of the bone screw 20 can engage the inner walls of the bore 52 to secure the suture anchor within the bore 52. One skilled in the art will appreciate that if a bore is not used, for example if the bone screw 20 is self threading, then the bone screw can engage the bone 54 as the bone screw 20 is advanced. Tension on the detached tissue 100 can be adjusted by driving the suture anchor 10 to a desired depth into the bone 54. As discussed above, the collar 30, including attachment points such as eyelet 32 and hook 34, can have an outer dimension less than the outer diameter of the helical thread 23. Such a configuration allows the collar 30 to be driven below the surface of a bone 54 without damaging the outer surface of the bone. Once the collar 30 is driven below the surface of the bone 54, it is prevented from rotating by interference between the bone 54 and the outer surfaces of the collar 30. When the anchor 10 has been inserted into bone 54 to the desired depth, the insertion tool 90 may be removed and the utility suture 75 can be removed and discarded. As shown in FIG. 10c, when the suture anchor 10 is properly advanced into the bone 54 there results a snug and anatomically correct attachment of the detached tissue 100 to the bone 54.

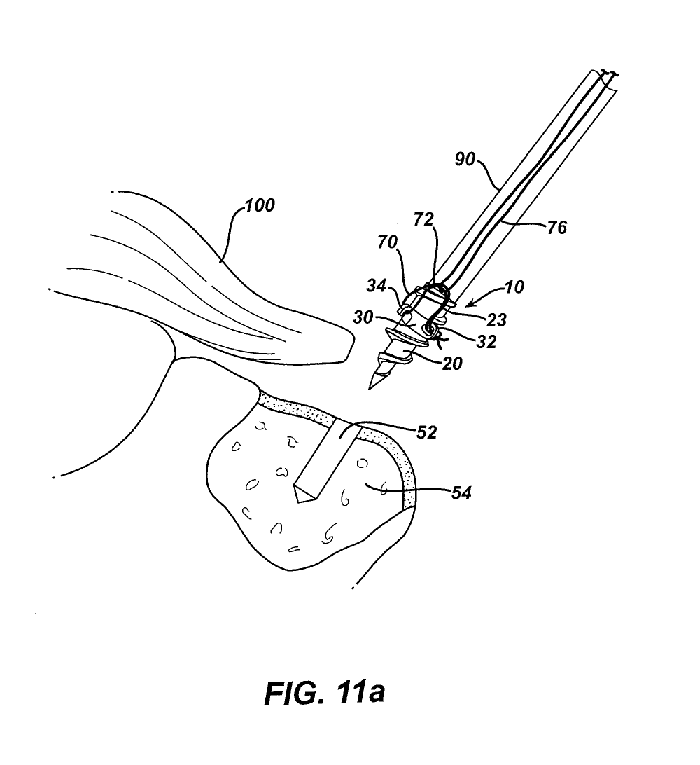

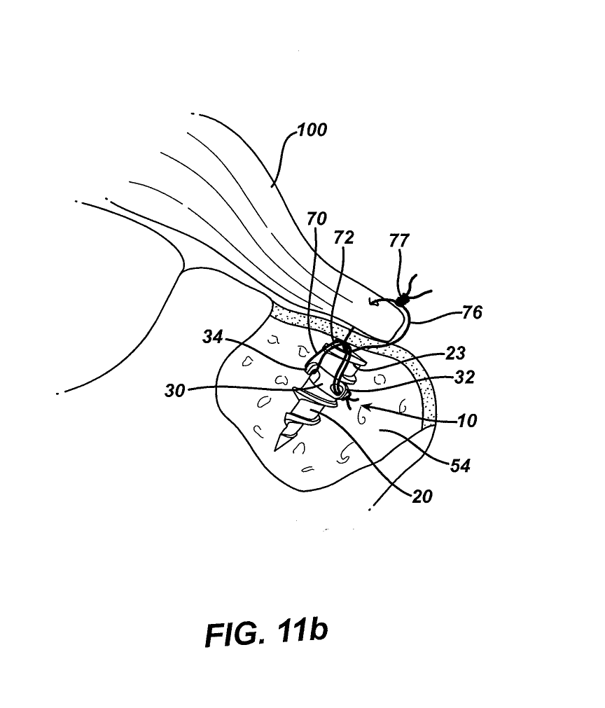

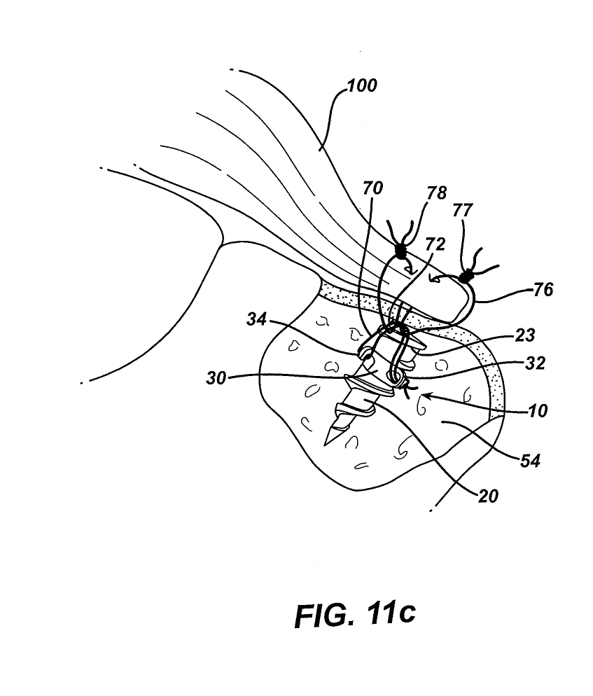

FIGS. 11a-11c show an alternative embodiment in which the suture loop 70 described herein can be used to create a suture eyelet that can then be used as the interface between the suture anchor and a strand of operative suture. Such a method is particularly useful with surgical procedures, both open and minimally invasive (e.g., arthroscopic), that require the tying of a knot with the operative suture to secure loose or torn tissue to a desired location to effect the surgical repair thereof.

As shown in FIG. 11a, an eyelet of suture 72 can be formed by pre-attaching the suture loop 70 to the hook 34 on the collar 30. As discussed above, the suture loop 70 can be provided separately from the suture anchor 10 or, alternatively, the anchor 10 may be provided with a suture loop pre-attached to the anchor. In either case, a set of suture loops of varying lengths can be provided with the suture anchor to allow a surgeon to select the desired length of suture loop (and suture eyelet formed therefrom) for a given procedure or patient. A surgeon can also form a suture loop of a desired length using suture not provided with the suture anchor. The suture eyelet 72 formed by the pre-attached suture loop 70 provides an interface with an operative suture strand 76 by interlocking therewith. For example, the operative suture 76 can be threaded through the pre-hooked suture loop 70. The operative suture strand 76 has two free ends (not shown) each of which may have a suture needle (not shown) attached thereto.

The surgical procedure can begin by forming a minimally invasive percutaneous incision through the tissue located adjacent to the desired surgical site. One skilled in the art will readily appreciate that the location, shape, and size of the incision will depend on the nature of the procedure, the patient's anatomy, and/or the preference of the surgeon. Following the formation of an incision to provide access to the surgical site, an optional bore 52 can be formed in a bone 54, as shown in FIG. 11a. As discussed above, one skilled in the art will appreciate that the anchor 10 can also be inserted into bone without the need for a bore to be formed in the bone.

To reach the configuration shown in FIG. 11b, the anchor 10 is advanced by rotation into the bone 54, for example, into a bore 52. During insertion of the suture anchor 10, the operative suture strand 76 can be used to provide tension on the suture eyelet 72 which prevents the collar 30 from rotating even though the bone screw is being rotated for insertion. Limiting, eliminating, or controlling rotation of the collar 30 with respect to the bone screw 20 can be desirable as it avoids tangling of the suture eyelet 72 and/or wrapping the suture eyelet 72 or the operative suture strand 76 around the shaft of the bone screw 20 during insertion. As the anchor 10 is driven below the surface of the bone 54, the suture eyelet 72 will become trapped between the threads of the bone screw and the bone 54. For example, if a bore 52 is used, the suture loop will become trapped between the threads of the bone screw 20 and the walls of the bore 52. As discussed above, a portion of the crest of the helical thread 23 proximal to the collar 30 can be blunt to avoid damage to the suture eyelet 72 as the bone screw 20 rotates into the bone 54. When the anchor 10 has been driven to the desired depth, the operative suture strand 76 can be used to approximate the detached tissue 100 to the bone 54. For example, the operative suture strand 76 can be passed through the detached tissue 100 using a needle (not shown) that may be attached to the free ends of the operative suture strand (and/or a separate tool). Those skilled in the art will appreciate that other methods of passing the operative suture strand 76 through the detached tissue 100 can be used depending upon the desired surgical application. The tissue repair is then completed by securing the tissue 100 in a desired location, such as by forming a knot 77 in the operative suture strand 76. If the procedure is being performed arthroscopically, the operative suture 76 and the tool with which it is associated will be operated through an exit portal (not shown).

In another exemplary embodiment, two or more operative suture strands can be used to approximate detached tissue to bone. For example, as shown in FIG. 11c, two operative suture strands 77, 78 can be threaded through the suture eyelet 72 for approximating the detached tissue 100 to the bone 54. Each of the two or more operative suture strands 77, 78 can be passed through the detached tissue 100 using needles (not shown) that may be attached to the free ends of each respective suture strand. Such a configuration can provide an additional attachment point to more securely anchor the detached tissue.

A particular advantage of the suture eyelet system described above and illustrated in FIGS. 11a-11c is that the length of the eyelet 72 is relatively small, enabling it to remain entirely beneath the surface of the bone. For example, if a bore is used, then the eyelet 72 can remain entirely within the bore formed in bone to accept the anchor. Such a system exposes the patient to less trauma and presents a more simple sliding interface between the operative suture strand and the flexible suture eyelet. For example, as shown in FIGS. 11a and 11b, the overall length of the suture loop 70 can be short enough so that when the suture anchor 10 is operatively disposed in a bore 52 formed in the bone 54 of a patient and the suture loop 70 is engaged with the suture anchor 10 to form a suture eyelet 72, the proximal-most portion of the suture eyelet 72 can be fully disposed within the bore 52. Those skilled in the art will readily appreciate that the length of the suture loop 70 and the corresponding length of the suture eyelet 72 may vary depending upon the desired surgical applications. For example, the length of the suture loop 70 and the suture eyelet 72 formed therefrom will typically depend on the nature of the procedure and/or the patient's anatomy.

A person skilled in the art will appreciate that the various methods, systems, and devices disclosed herein can be formed from a variety of materials. Moreover, particular components can be implantable and in such embodiments the components can be formed from various biocompatible materials known in the art. Exemplary biocompatible materials include, by way of non-limiting example, composite materials, polymeric materials, biocompatible metals and alloys such as stainless steel, titanium, titanium alloys and cobalt-chromium alloys, and any other material that is biologically compatible and non-toxic to the human body.

One skilled in the art will appreciate further features and advantages based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1-18. (canceled)

19. A method for anchoring tissue to bone, comprising:
providing a suture anchor having a collar rotatably disposed thereon with a suture loop pre-attached to a portion of the collar and a utility suture attached to the suture loop;
passing the utility suture through a detached segment of tissue;
manipulating the suture anchor to engage a portion of the suture loop within a portion of the collar; and
rotating the suture anchor to implant the suture anchor into bone while tensioning the suture loop to prevent the collar from rotating with respect to the suture anchor such that the suture loop reattaches the detached segment to bone.

20. The method of claim 19, wherein a needle is attached to the utility suture.

21. The method of claim 19, wherein the suture loop is pre-attached to an eyelet on the collar.

22. The method of claim 19, wherein the suture loop is engaged with a hook portion of the collar.
